(19) United States
(12) Patent Application Publication
(10) Pub. No.: US 2005/0009927 A1
(43) Pub. Date: Jan. 13, 2005

(54) Combination of serotonin reuptake inhibitors and norepinephrine reuptake inhibitors

(75) Inventors: Gerard J. Marek, Indianapolis, IN (US); Earl L. Giller, Madison, CT (US); Tanya S. Ramey, New London, CT (US); Megan A. Gibbs, Noank, CT (US); Erik H. F. Wong, Portage, MI (US); Robert C. Marshall, Mattawan, MI (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/860,721

(22) Filed: Jun. 3, 2004

Related U.S. Application Data
(63) Continuation-in-part of application No. 10/055,663, filed on Jan. 23, 2002.
(63) Continuation-in-part of application No. 10/602,447, filed on Jun. 24, 2003.
(60) Provisional application No. 60/501,275, filed on Sep. 9, 2003.
(60) Provisional application No. 60/538,898, filed on Jan. 23, 2004.
(60) Provisional application No. 60/540,696, filed on Jan. 30, 2004.
(60) Provisional application No. 60/392,893, filed on Jul. 1, 2002.

(51) Int. Cl. A61K 31/137

(52) U.S. Cl. 514/651

(57) ABSTRACT

This invention is directed in one embodiment to pharmaceutical compositions and methods for treating depression in a mammal. To a mammal in need of such treatment are administered: (i) at least one serotonin reuptake inhibitor or pharmaceutically acceptable salt thereof; and (ii) at least one norepinephrine reuptake inhibitor or pharmaceutically acceptable salt thereof, wherein the norepinephrine reuptake inhibitor is selected from the group consisting of Structure II, Structure III, and Structure IV as defined herein.

### CROSS-REFERENCE TO RELATED APPLICATIONS

This application claims priority from U.S. Provisional Applications Ser. Nos. 60/501,275, filed Sep. 9, 2003, 60/538,898, filed Jan. 23, 2004, and 60/540,696, filed Jan. 30, 2004, each of which is incorporated by reference herein in its entirety; and is a continuation-in-part of U.S. Ser. No. 10/055,663, filed Jan. 23, 2002, which claims priority from U.S. Ser. Nos. 60/141,968, filed Jul. 1, 1999, 60/144,131, filed Jul. 16, 1999, 60/158,256, filed Oct. 6, 1999, and 60/170,381, filed Dec. 13, 1999, each of which is incorporated by reference herein in its entirety; and is a continuation-in-part of U.S. Ser. No. 10/602,447, filed Jun. 24, 2003, which claims priority from U.S. Ser. No. 60/392, 893, filed Jul. 1, 2002, each of which is incorporated by reference herein in its entirety.

### BACKGROUND OF THE INVENTION

This invention is directed to a pharmaceutical compositions comprising a serotonin reuptake inhibitor or pharmaceutically acceptable salts thereof and a norepinephrine reuptake inhibitor or pharmaceutically acceptable salts thereof, and to methods of treatment with such a composition.

This invention is also directed to a pharmaceutical composition comprising a serotonin reuptake inhibitor or pharmaceutically acceptable salts thereof and optionally a norepinephrine reuptake inhibitor or pharmaceutically acceptable salts thereof, and to methods of treatment with such a composition, wherein the serotonin reuptake inhibitor is present in an amount sufficient for dopamine reuptake inhibition.

Serotonin plays a role in several psychiatric disorders, including anxiety, Alzheimer's disease, depression, nausea and vomiting, eating disorders, and migraine (see Rasmussen et al., “Chapter 1. Recent Progress in Serotonin (5HT)_{1A} Receptor Modulators”, in Annual Reports in Medicinal Chemistry, Section I, 30, pp. 1-9, 1995, Academic Press, Inc.; Artigas et al., Trends Neurosci., 19 (9), 1996, pp. 378-383; and Wolf et al., Drug Development Research, 40, 1997, pp. 17-34). Serotonin also plays a role in both the positive and negative symptoms of schizophrenia, as discussed in Sharma et al., Psychiatric Annals., 26 (2), February, 1996, pp. 88-92. Serotonin reuptake inhibitors, have been used to treat disorders or conditions such as depression, as described, for example, in WO 94/00047.

The antidepressant effect of norepinephrine reuptake inhibitors has been described, for example, in U.S. Pat. No. 6,403,645.

The effect of combining selective serotonin reuptake inhibitors (SSRIs) in depressed patients with the norepinephrine reuptake inhibitor desipramine has been described in J. C. Nelson et al., Arch. Gen. Psychiatry, 39: 1419-1422, 1982; J. C. Nelson et al., Arch. Gen. Psychiatry, 48: 303-307, 1991; J. C. Nelson and L. H. Price, Am. J. Psychiatry 152: 1538-1539, 1995; and M. Fava et al., Am. J. Psychiatry 152: 1539, 1995. A combination of sertraline and of reboxetine has been described in Eur J Pharmacol, 1999, 364(2-3):123-32 and in J. Neural Transm., 2000, 107:1213-1227. A comparison of the stimulus properties of reboxetine and certain serotonin reuptake inhibitors has been described in Pychopharmacology 2001, 154:213-218. A combination of citalopram and of reboxetine has been described in WO 02/076461.

According to Brunswick et al., Am. J. Psychiatry, 2003, Vol. 160:10, pp. 1836-1841, dopamine transporter affinity may be higher than normal in the basal ganglia of depressed patients. Racemic reboxetine, a norepinephrine reuptake inhibitor, has been shown to affect the dopaminergic system preclinically, increasing dopamine in the prefrontal cortex. See Sacchetti et al., Br. J. Pharmacol. 1999, Vol. 128, pp. 1332-1338; Page et al, Neuropsychopharmacology, 2002, Vol. 27, pp. 237-247; and Invernizzi et al., British J. of Pharmacology, 2001, Vol.132, pp.183-188.

However, citalopram, a serotonin reuptake inhibitor, does not show dopamine reuptake inhibition. Kugaya et al., Neuropsychopharmacology, 2003, Vol. 28, pp. 413-420. Similarly, the serotonin and norepinephrine reuptake inhibitor venlafaxine does not show dopamine reuptake inhibition (Marek et al, Society for Neuroscience Meeting, New Orleans, November 2003).

None of the previously published studies discloses or suggests a composition having a combined action of serotonin reuptake inhibition, norepinephrine reuptake inhibition, and dopamine reuptake inhibition in a dual component therapy, or methods of treatment with such a composition.

### SUMMARY OF THE INVENTION

This invention is directed to a pharmaceutical composition for treating a disorder or condition selected from the group consisting of anxiety disorders, phobias, avoidant personality disorder, eating disorders, chemical dependencies, Parkinson's diseases, obsessive-compulsive disorder, negative symptoms of schizophrenia, cognitive dysfunction related to schizophrenia, premenstrual syndrome, stress-induced incontinence, headache, neuropathic pain, chronic pain, urinary incontinence, fibromyalgia, depression comorbid with fibromyalgia, obesity, migraine, neuropathic pain associated with diabetes, affective symptoms of schizophrenia and a combination thereof in a mammal, the composition comprising: (i) at least one serotonin reuptake inhibitor or pharmaceutically acceptable salt thereof; (ii) at least one norepinephrine reuptake inhibitor or pharmaceutically acceptable salt thereof, wherein the at least one norepinephrine reuptake inhibitor is selected from the group consisting of

(A) a compound having the formula of Structure II 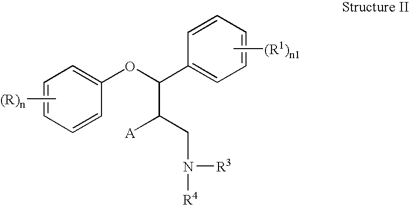
wherein n and n₁ are, independently, 1, 2 or 3; each of the groups R and R¹, which may be the same or different, is hydrogen; halogen; halo-C₁-C₆ alkyl; hydroxy; C₁-C₆ alkoxy; C₁-C₆ alkyl optionally substituted with C₁-C₆ alkyl or halogen; aryl- C₁-C₆ alkyl optionally substituted with C₁-C₆ alkyl or halogen; aryl-C₁-C₆ alkoxy optionally substituted with C₁-C₆ alkyl or halogen; —NO₂; —NR⁵R⁶ wherein R⁵ and R⁶ are, independently, hydrogen or C₁-C₆ alkyl, or two adjacent R groups or two adjacent R¹ groups, taken together, form the —O—CH₂—O— group;

A is hydrogen or OR²;

R is hydrogen; C₁-C₁₂ alkyl optionally substituted with C₁-C₆ alkyl or halogen, or aryl-C₁-C₆ alkyl;

each of the groups R³ and R⁴, which may be identical or different, is hydrogen, C₁-C₆ alkyl optionally substituted with C₁-C₆ alkyl or halogen, C₂-C₄ alkenyl, C₂-C₄ alkynyl, aryl-C₁-C₄ alkyl optionally substituted with C₁-C₆ alkyl or halogen, C₃-C₇ cycloalkyl optionally substituted with C₁-C₆ alkyl or halogen, or R³ and R⁴with the nitrogen atom to which they are bound form a pentatomic or hexatomic saturated or unsaturated, optionally substituted with C₁-C₆ alkyl or halogen, heteromonocyclic group optionally containing other heteroatoms selected from the group consisting of O,S and N;

or R² and R⁴, taken together, form the —CH₂—H₂-group;

(B) a compound having the formula of Structure III 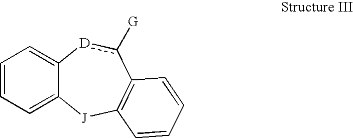
wherein D is N or CR⁹, where R⁹ is hydrogen, C₁-C₆ alkyl optionally substituted with C₁-C₆ alkyl or halogen, C₂-C₄ alkenyl, C₂-C₄ alkynyl, aryl-C₁-C₄ alkyl optionally substituted with C₁-C₆ alkyl or halogen, or C₃-C₇ cycloalkyl optionally substituted with C₁-C₆ alkyl or halogen; G is NR⁷R⁸, wherein each of R⁷ and R⁸ is independently hydrogen, C₁-C₆ alkyl optionally substituted with C₁-C₆ alkyl or halogen, C₂-C₄ alkenyl, C₂-C₄ alkynyl, aryl-C₁-C₄ alkyl optionally substituted with C₁-C₆ alkyl or halogen, or C₃-C₇ cycloalkyl optionally substituted with C₁-C₆ alkyl or halogen; or R⁷ and R⁸ taken together with the nitrogen atom to which they are bound form a pentatomic or hexatomic, saturated or unsaturated, optionally substituted with C₁-C₆ alkyl or halogen heteromonocyclic group optionally containing one or more further additional heteroatoms selected from the group consisting of O,S and N; the bond between D and the ring carbon bonded to G is single or double; and J is O or L, where L is 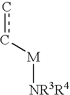
where the bond between the ring carbon of L and the carbon of L bonded to M is single or double; M is a Cₙ alkylene chain, where n is between 1 and 3; and each of R¹³ and R¹⁴ is independently hydrogen, C₁-C₆ alkyl optionally substituted with C₁-C₆ alkyl or halogen, C₂-C₄ alkenyl, C₂-C₄ alkynyl, aryl-C₁-C₄ alkyl optionally substituted with C₁-C₆ alkyl or halogen, C₃-C₇ cycloalkyl optionally substituted with C₁-C₆ alkyl or halogen, or R¹³ and R¹⁴ taken together with the nitrogen atom to which they are bound form a pentatomic or hexatomic, saturated or unsaturated, optionally substituted with C₁-C₆ alkyl or halogen heteromonocyclic group optionally containing one or more further additional heteroatoms selected from the group consisting of O,S and N; and

(C) a compound having the formula of Structure IV: 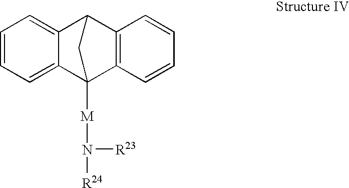
wherein M is a Cₙ alkylene chain, where n is between 1 and 3; and each of R²³ and R²⁴ is independently hydrogen, C₁-C₆ alkyl optionally substituted with C₁-C₆ alkyl or halogen, C₂-C₄ alkenyl, C₂-C₄ alkynyl, aryl-C₁-C₄ alkyl optionally substituted with C₁-C₆ alkyl or halogen, C₃-C₇ cycloalkyl optionally substituted with C₁-C₆ alkyl or halogen, or R²³ and R²⁴taken together with the nitrogen atom to which they are bound form a pentatomic or hexatomic, saturated or unsaturated, optionally substituted with C₁-C₆ alkyl or halogen heteromonocyclic group optionally containing one or more further additional heteroatoms selected from the group consisting of O,S and N;
and

(iii) a pharmaceutically acceptable carrier.

This invention is also directed to:

a method for treating a disorder or condition described in the previous paragraph in a mammal, the method comprising administering to a mammal in need of such treatment components (i) and (ii) described in the previous paragraph;

the use of components (i) and (ii) described in the previous paragraph for preparing a medicament for treating a disorder or condition described in the previous paragraph in a mammal;

a pharmaceutical composition for treating a disorder or condition that can be treated by enhancing serotonergic neurotransmission in a mammal, noradrenergic neurotransmission in a mammal, or a combination thereof, the composition comprising components (i), (ii) and (iii) described in the previous paragraph;

a method for treating a disorder or condition that can be treated by enhancing. serotonergic neurotransmission in a mammal, noradrenergic neurotransmission in a mammal, or a combination thereof, the method comprising administering to a mammal in need of such treatment components (i) and (ii) described in the previous paragraph;

a pharmaceutical composition for treating depression in a mammal, the composition comprising components (i), (ii) and (iii) described in the previous paragraph;

a method for treating depression in a mammal, the method comprising administering to a mammal in need of such treatment components (i) and (ii) described in the previous paragraph; and

the use of components (i) and (ii) described in the previous paragraph for preparing a medicament for treating depression in a mammal.

This invention is also directed to a composition for treating a disorder or condition described in the previous paragraphs in a mammal, the composition consisting essentially of components (i), (ii) and (iii) described in the previous paragraphs.

This invention is also directed to:

a composition comprising (i) at least one serotonin reuptake inhibitor or pharmaceutically acceptable salt thereof, wherein the at least one serotonin reuptake inhibitor is selected from the group consisting of sertraline, fluoxetine and fluvoxamine; and (ii) at least one norepinephrine reuptake inhibitor or pharmaceutically acceptable salt thereof, wherein the at least one norepinephrine reuptake inhibitor is selected from the group consisting of racemic reboxetine, [S,S]-reboxetine, amoxapine, and maprotiline. The composition may be a composition for treating a disorder or condition selected from the group consisting of neuropathic pain, chronic pain, urinary incontinence, fibromyalgia, depression comorbid with fibromyalgia, obesity, migraine, neuropathic pain associated with diabetes, affective symptoms of schizophrenia and a combination thereof in a mammal; and

a composition consisting essentially of (i) at least one serotonin reuptake inhibitor or pharmaceutically acceptable salt thereof, wherein the at least one serotonin reuptake inhibitor is selected from the group consisting of sertraline, fluoxetine and fluvoxamine; and (ii) at least one norepinephrine reuptake inhibitor or pharmaceutically acceptable salt thereof, wherein the at least one norepinephrine reuptake inhibitor is selected from the group consisting of racemic reboxetine, [S,S]-reboxetine, amoxapine, and maprotiline. The composition may be a composition for treating a disorder or condition selected from the group consisting of neuropathic pain, chronic pain, urinary incontinence, fibromyalgia, depression comorbid with fibromyalgia, obesity, migraine, neuropathic pain associated with diabetes, affective symptoms of schizophrenia and a combination thereof in a mammal.

The invention is also directed to:

a method for treating a disorder or condition described in the previous paragraphs in a mammal, the method comprising administering to a mammal in need of such treatment (i) at least one serotonin reuptake inhibitor or pharmaceutically acceptable salt thereof, wherein the at least one serotonin reuptake inhibitor is selected from the group consisting of sertraline, fluoxetine and fluvoxamine; and (ii) at least one norepinephrine reuptake inhibitor or pharmaceutically acceptable salt thereof, wherein the at least one norepinephrine reuptake inhibitor is selected from the group consisting of racemic reboxetine, [S,S]-reboxetine, amoxapine, and maprotiline; and

the use of components (i) and (ii) described in the previous paragraphs for preparing a medicament for treating a disorder or condition described in the previous paragraphs in a mammal.

The invention is also directed to a composition comprising sertraline or a pharmaceutically acceptable salt thereof and [S,S]-reboxetine or a pharmaceutically acceptable salt thereof.

The invention is also directed to a composition for treating, for example, a disorder or condition that can be treated by enhancing serotonergic neurotransmission in a mammal, dopaminergic transmission in a mammal, noradrenergic neurotransmission in a mammal, or a combination thereof, the composition comprising component (i) and optionally component (ii) described in the previous paragraphs, wherein component (i) is present in an amount sufficient for dopamine reuptake inhibition. Preferably, component (i) is sertraline or a pharmaceutically acceptable salt thereof.

The invention is also directed to a method for treating a disorder or condition that can be treated by enhancing serotonergic neurotransmission in a mammal, dopaminergic transmission in a mammal, noradrenergic neurotransmission in a mammal, or a combination thereof, the method comprising administering to a mammal in need of such treatment comprising component (i) and optionally component (ii) described in the previous paragraphs, wherein component (i) is present in an amount sufficient for dopamine reuptake inhibition. Preferably, component (i) is sertraline or a pharmaceutically acceptable salt thereof.

The invention is also directed to a composition comprising sertraline or a pharmaceutically acceptable salt thereof and optionally a norepinephrine reuptake inhibitor selected from the group consisting of racemic reboxetine, [S,S]-reboxetine, amoxapine, and maprotiline, or pharmaceutically acceptable salts thereof, wherein sertraline or a pharmaceutically acceptable salt thereof is present in an amount sufficient for dopamine reuptake inhibition.

The invention is also directed to a composition comprising sertraline or a pharmaceutically acceptable salt thereof and optionally [S,S]-reboxetine or a pharmaceutically acceptable salt thereof, wherein sertraline is present in an amount sufficient for dopamine reuptake inhibition.

In the method for treating a disorder or condition selected from the group consisting of anxiety disorders, phobias, avoidant personality disorder, eating disorders, chemical dependencies, Parkinson's diseases, obsessive-compulsive disorder, negative symptoms of schizophrenia, premenstrual syndrome, stress-induced incontinence, headache, neuropathic pain, chronic pain, urinary incontinence, post-traumatic stress disorder, chronic stress, acute stress, post-traumatic stress disorder, fibromyalgia, depression comorbid with fibromyalgia, obesity, the serotonin reuptake inhibitor may be sertraline present in an amount sufficient for dopamine reuptake inhibition. Similarly, in the composition for treating a disorder or condition as recited in this paragraph, the serotonin reuptake inhibitor may be sertraline present in an amount sufficient for dopamine reuptake inhibition. Similarly, in the method for treating a disorder or condition selected from the group consisting of neuropathic pain, chronic pain, urinary incontinence, post-traumatic stress disorder, chronic stress, acute stress, post-traumatic stress disorder, fibromyalgia, depression comorbid with fibromyalgia, obesity, the serotonin reuptake inhibitor may be sertraline present in an amount sufficient for dopamine reuptake inhibition.

In the method for treating depression, the serotonin reuptake inhibitor may be sertraline or a pharmaceutically salt thereof present in an amount sufficient for dopamine reuptake inhibition. In one embodiment, the serotonin reuptake inhibitor is sertraline or a pharmaceutically acceptable salt thereof, and the norepinephrine reuptake inhibitor is [S,S]-reboxetine or a pharmaceutically acceptable salt thereof, wherein sertraline is present in an amount sufficient for dopamine reuptake inhibition, wherein the amount of sertraline is from about 150 mg to about 200 mg.

Similarly, in the composition for treating depression, the serotonin reuptake inhibitor may be sertraline present in an amount sufficient for dopamine reuptake inhibition. In one embodiment, the serotonin reuptake inhibitor is sertraline or a pharmaceutically acceptable salt thereof, and the norepinephrine reuptake inhibitor is [S,S]-reboxetine or a pharmaceutically acceptable salt thereof, wherein sertraline is present in an amount sufficient for dopamine reuptake inhibition, wherein the amount of sertraline is from about 150 mg to about 200 mg.

In the composition comprising (i) at least one serotonin reuptake inhibitor or pharmaceutically acceptable salt thereof, wherein the at least one serotonin reuptake inhibitor is selected from the group consisting of sertraline, fluoxetine and fluvoxamine; and (ii) at least one norepinephrine reuptake inhibitor or pharmaceutically acceptable salt thereof, wherein the at least one norepinephrine reuptake inhibitor is selected from the group consisting of racemic reboxetine, [S,S]-reboxetine, amoxapine, and maprotiline, the serotonin reuptake inhibitor may be sertraline present in an amount sufficient for dopamine reuptake inhibition. Similarly, in the composition consisting essentially of (i) at least one serotonin reuptake inhibitor or pharmaceutically acceptable salt thereof, wherein the at least one serotonin reuptake inhibitor is selected from the group consisting of sertraline, fluoxetine and fluvoxamine; and (ii) at least one norepinephrine reuptake inhibitor or pharmaceutically acceptable salt thereof, wherein the at least one norepinephrine reuptake inhibitor is selected from the group consisting of racemic reboxetine, [S,S]-reboxetine, amoxapine, and maprotiline, the serotonin reuptake inhibitor may be sertraline present in an amount sufficient for dopamine reuptake inhibition.

The pharmaceutical compositions and methods of this invention may also be used for preventing a relapse in a disorder or condition described in the previous paragraphs by administering to a mammal in need of such prevention components (i) and (ii) of the composition. The pharmaceutical compositions and methods of this invention may further be used for treating a symptom associated with a disorder or condition described in the previous paragraphs, wherein the symptom is selected from the group consisting of cognitive dysfunctions and somatic complaints.

“Enhancing serotonergic neurotransmission,” as used herein, refers to increasing or improving the neuronal process whereby the monoamine serotonin is released by a pre-synaptic cell upon excitation and crosses the synapse to stimulate or inhibit the post-synaptic cell. “Enhancing dopaminergic neurotransmission,” as used herein, refers to increasing or improving the neuronal process whereby the monoamine dopamine is released by a pre-synaptic cell upon excitation and crosses the synapse to stimulate or inhibit the post-synaptic cell. “Enhancing noradrenergic neurotransmission,” as used herein, refers to increasing or improving the neuronal process whereby the monoamine norepinephrine is released by a pre-synaptic cell upon excitation and crosses the synapse to stimulate or inhibit the post-synaptic cell.

“Chemical dependency,” as used herein, means an abnormal craving or desire for, or an addiction to a drug. Such drugs are generally administered to the affected individual by any of a variety of means of administration, including oral, parenteral, nasal or by inhalation. “Treating a chemical dependency,” as used herein, means reducing or alleviating such dependency.

A “unit dosage form” as used herein is any form that contains a unit dose of the serotonin reuptake inhibitor or a pharmaceutically acceptable salt thereof, of the norepinephrine reuptake inhibitor or a pharmaceutically acceptable salt thereof, or of the serotonin reuptake inhibitor or pharmaceutically acceptable salt thereof and the norepinephrine reuptake inhibitor or pharmaceutically acceptable salt thereof. A unit dosage form may be, for example, a tablet or a capsule. The unit dosage form may also be in liquid form, such as a solution or suspension.

A “serotonin reuptake inhibitor” as used herein is a reuptake inhibitor of the monoamine serotonin. For example, the serotonin reuptake inhibitor may be a selective serotonin reuptake inhibitor (SSRI). The serotonin reuptake inhibitor may have additional pharmacological properties, for example, antagonism of 5-HT_{1A} or 5-HT_{2A/C} receptors, or partial inhibition of a norepinephrine transporter (NET). At or above certain doses, as: described further herein, the serotonin reuptake inhibitor shows dopamine reuptake inhibition.

“An amount sufficient for dopamine reuptake inhibition” is intended to refer to an amount that is sufficient to displace at least about 15% β-CIT from the dopamine transporter as assessed by SPECT imaging.

Exemplary selective serotonin reuptake inhibitors (SSRI) which may be used in accordance with this invention include those having structure I shown below: 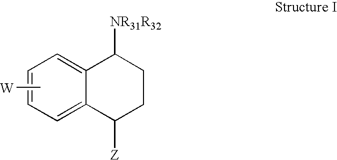
or a pharmaceutically acceptable salt thereof, wherein R₃₁ is selected from the group consisting of hydrogen and normal alkyl of from 1 to 3 carbon atoms, R₃₂ is normal alkyl of from 1 to 3 carbon atoms, Z is

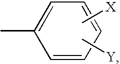

X and Y are each selected from the group consisting of hydrogen, fluoro, chloro, bromo, trifluoromethyl, alkoxy of from 1 to 3 carbon atoms and cyano, with at least one of X and Y being other than hydrogen,

W is selected from the group consisting of hydrogen, fluoro, chloro, bromo, trifluoromethyl and alkoxy of from 1 to 3 carbon atoms,

and NR₃₁R₃₂ and Z have a cis relationship.

An exemplary compound of Structure I is sertraline, (1S-cis)-4-(3,4-dichlorophenyl)-1,2,3,4-tetrahydro-N-methyl-1-naphthalenamine, which may be prepared as described in U.S. Pat. No. 4,536,518. SSRI that may be used in accordance with this invention also include, but are not limited to: femoxetine, which may be prepared as described in U.S. Pat. No. 3,912,743; fluoxetine, which may be prepared as described in U.S. Pat. No. 4,314,081; fluvoxamine, which may be prepared as described in U.S. Pat. No. 4,085,225; indalpine, which may be prepared as described in U.S. Pat. No. 4,064,255; indeloxazine, which may be prepared as described in U.S. Pat. No. 4,109,088; milnacipran, which may be prepared as described in U.S. Pat. No. 4,478,836; paroxetine, which may be prepared as described in U.S. Pat. No. 3,912,743 or U.S. Pat. No. 4,007,196; sibutramine, which may be prepared as described in U.S. Pat. No. 4,929,629; zimeldine, which may be prepared as described in U.S. Pat. No. 3,928,369; citalopram; escitalopram; fenfluramine; venlafaxine and duloxetine.

A “norepinephrine reuptake inhibitor” as used herein is a reuptake inhibitor of the monoamine norepinephrine. For example, the norepinephrine reuptake inhibitor may be a selective norepinephrine reuptake inhibitor. As another example, the norepinephrine reuptake inhibitor may have additional pharmacological properties, for example, antagonism of 5-HT₂ receptors. As another example, the norepinephrine reuptake inhibitor may affect the dopaminergic tranport system, for example, through the intermediacy of NET. Norepinephrine reuptake inhibition is readily determined by those skilled in the art according to standard assays. As an example, norepinephrine reuptake inhibitors which may be used in accordance with this invention include compounds having structure II defined above, or a pharmaceutically acceptable salt thereof. As another example, norepinephrine reuptake inhibitors which may be used in accordance with this invention include compounds having structure III defined above, or a pharmaceutically acceptable salt thereof. As another example, norepinephrine reuptake inhibitors which may be used in accordance with this invention include compounds having structure IV defined above, or a pharmaceutically acceptable salt thereof.

In an exemplary embodiment of the invention, the norepinephrine reuptake inhibitor is the [S,S]-enantiomer of Structure II, shown as Structure II′ below, where A=OR² and R, R¹, R², R³, R⁴, n and n1 are as defined in Structure II:

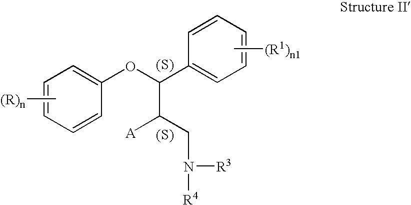

For example, the norepinephrine reuptake inhibitor of Structure II′ may be [S,S]-reboxetine, which is described in British patent GB 2,167,407, U.S. Patent Application No. 20020061910, U.S. Pat. No. 6,465,458.

In another exemplary embodiment of the invention, the norepinephrine reuptake inhibitor of Structure II is atomoxetine or racemic reboxetine.

In another exemplary embodiment of the invention, the norepinephrine reuptake inhibitor of Structure III is amoxapine, nortriptyline, or protriptyline.

In another exemplary embodiment of the invention, the norepinephrine reuptake inhibitor of Structure IV is maprotiline.

In one exemplary embodiment of the composition of the invention, the composition comprises a norepinephrine reuptake inhibitor having Structure II′, and sertraline as the serotonin reuptake inhibitor.

In another exemplary embodiment of the invention, the composition comprises sertraline as the serotonin reuptake inhibitor and [S,S]-reboxetine as the norepinephrine reuptake inhibitor.

In another exemplary embodiment of the invention, the composition consists essentially of sertraline as the serotonin reuptake inhibitor and [S,S]-reboxetine as the norepinephrine reuptake inhibitor.

The combination of a norepinephrine reuptake inhibitor or a pharmaceutically acceptable salt thereof and a serotonin reuptake inhibitor or pharmaceutically acceptable salt thereof is also referred to herein as “the active combination.” The term “the active combination” may also be used to denote the combination of a norepinephrine reuptake inhibitor or a pharmaceutically acceptable salt thereof and a serotonin reuptake inhibitor or pharmaceutically acceptable salt thereof wherein the serotonin reuptake inhibitor or pharmaceutically acceptable salt also inhibits dopamine reuptake. The active combination is a useful psychotherapeutic and may be used in the treatment of the disorders or conditions described herein. Examples of the disorders or conditions which may be treated by the methods and compositions of this invention are as follows:

depression, including, for example, depression in cancer patients, depression in Parkinson's patients, Postmyocardial Infarction depression, depression in patients with human immunodeficiency virus (HIV), Subsyndromal Symptomatic depression, depression in infertile women, pediatric depression, major depression, single episode depression, recurrent depression, child abuse induced depression, post partum depression, DSM-IV major depression, treatment-refractory major depression, severe depression, psychotic depression, post-stroke depression, neuropathic pain, manic depressive illness, including manic depressive illness with mixed episodes and manic depressive illness with depressive episodes, seasonal affective disorder, bipolar depression BP I, bipolar depression BP II, melancholy, or major depression with dysthymia; dysthymia; anxiety disorders, including, for example, generalized anxiety disorder, panic disorder, PTSD, and social anxiety disorder; phobias, including, for example, agoraphobia, social phobia or simple phobias; eating disorders, including, for example, anorexia nervosa or bulimia nervosa; chemical dependencies, including, for example, addictions to alcohol, cocaine, amphetamine and other psychostimulants, morphine, heroin and other opioid agonists, phenobarbital and other barbiturates, nicotine, diazepam, benzodiazepines and other psychoactive substances; Parkinson's diseases, including, for example, dementia in Parkinson's disease, neuroleptic-induced parkinsonism or tardive dyskinesias; and headache, including, for example, headache associated with vascular disorders.

Disorders or conditions that may be treated by the composition and method of the present invention also include withdrawal syndrome; adjustment disorders, including depressed mood, mixed anxiety and depressed mood, disturbance of conduct, and mixed disturbance of conduct and depressed mood; age-associated learning and mental disorders, including Alzheimer's disease; apathy; attention-deficit disorders, or other cognitive disorders, due to general medical conditions; attention-deficit hyperactivity disorder (ADHD); bipolar disorder; chronic fatigue syndrome; chronic or acute stress; conduct disorder; cyclothymic disorder; somatoform disorders such as somatization disorder, conversion disorder, pain disorder, hypochondriasis, body dismorphic disorder, undifferentiated disorder, and somatoform NOS; incontinence; inhalation disorders; intoxication disorders; mania; oppositional defiant disorder; peripheral neuropathy; post-traumatic stress disorder; late luteal phase dysphoric disorder; psychotic disorders including schizoaffective disorders; sleep disorders, including narcolepsy and enuresis; specific developmental disorders; SSRI “poop out” syndrome, or a patient's failure to maintain a satisfactory response to SSRI therapy after an initial period of satisfactory response; and tic disorders including Tourette's disease.

As an example, the mammal in need of the treatment or prevention may be a human. As another example, the mammal in need of the treatment or prevention may be a mammal other than a human.

A norepinephrine reuptake inhibitor and a serotonin reuptake inhibitor, each of which is used in formulating the pharmaceutical composition of this invention, are each referred to herein as an “active compound.” An active compound which is basic in nature is capable of forming a wide variety of different salts with various inorganic and organic acids. The acid addition salts are readily prepared by treating the base compounds with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid salts of the active compounds used in formulating the pharmaceutical compositions of this invention that are basic in nature are those which form non-toxic acid addition salts, i.e., salts containing pharmacologically acceptable anions. Non-limiting examples of the salts include the acetate, benzoate, β-hydroxybutyrate, bisulfate, bisulfite, bromide, butyne-1,4-dioate, carpoate, chloride, chlorobenzoate, citrate, dihydrogenphosphate, dinitrobenzoate, fumarate, glycollate, heptanoate, hexyne-1,6-dioate, hydroxybenzoate, iodide, lactate, maleate, malonate, mandelate, metaphosphate, methanesulfonate, methoxybenzoate, methylbenzoate, monohydrogenphosphate, naphthalene-1-sulfonate, naphthalene-2-sulfonate, oxalate, phenylbutyrate, phenylproionate, phosphate, phthalate, phylacetate, propanesulfonate, propiolate, propionate, pyrophosphate, pyrosulfate, sebacate, suberate, succinate, sulfate, sulfite, sulfonate, tartrate, xylenesulfonate, acid phosphate, acid citrate, bitartrate, succinate, gluconate, saccharate, nitrate, methanesulfonate and pamoate [i.e., 1,1′-methylene-bis-(2-hydroxy-3-naphthoate)] salts. For the norepinephrine reuptake inhibitors, such as reboxetine and [S,S]-reboxetine, advantageous examples include the fumarate, succinate, citrate and tartrate salts.

### DETAILED DESCRIPTION OF THE INVENTION

The norepinephrine reuptake inhibitors and serotonin reuptake inhibitors, which may also show dopamine reuptake inhibition as described herein, that are used in formulating the pharmaceutical compositions of this invention are preferably administered together in a pharmaceutical composition. For example, compositions containing the serotonin reuptake inhibitors and the norepinephrine reuptake inhibitors can be administered as solutions in a volume of 1 ml/kg. The vehicle used is varied depending on the solubility of the serotonin reuptake inhibitor and of the norepinephrine reuptake inhibitor used.

The norepinephrine reuptake inhibitors and the serotonin reuptake inhibitors, which may also show dopamine reuptake inhibition as described herein, that are used in formulating the pharmaceutical compositions of this invention may advantageously be used in conjunction with other therapeutic agents which do not appreciably block serotonin uptake or affect monoamine oxidase, such as mirtazapine, mianserin, bupropion, lithium salts, antiepileptic drugs such as caramazepine, valproate, lamotrigine, topiramate, gabapentin, pregabalin, with atypical antipsychotic drugs such as olanzapine, risperidone, quetiapine, ziprasidone and aripiprazole, and/or with antiparkinsonian agents such as dopaminergic antiparkinsonian agents such as levodopa, preferably in combination with a peripheral decarboxylase inhibitor such as benserazide or carbidopa. As another example, the norepinephrine reuptake inhibitor and the serotonin reuptake inhibitor, which may also show dopamine reuptake inhibition as described herein, that are used in formulating the pharmaceutical composition of this invention may advantageously be used in combination with a 5-HT1B antagonist. An exemplary is elzasonan or a pharmaceutically acceptable salt thereof. For example, elzosonan may be present in amounts such as about 0.1 to about 200 mg, for example about 0.1 mg to about 50 mg, as another example from about 0.1 mg to about 20 mg, as another example from about 0.1 mg to about 10 mg, as another example from about 0.1 mg to about 5 mg. The methods of treatment of the invention likewise may include treatment of a disorder or condition with a norepinephrine reuptake inhibitor, a serotonin reuptake inhibitor, and a 5-HT1B antagonist such as elzasonan.

It is to be understood that the present invention covers the use of a serotonin reuptake inhibitor or a pharmaceutically acceptable salt thereof and a norepinephrine reuptake inhibitor or pharmaceutically acceptable salt thereof in combination with one or more other therapeutic agents.

Activity of the active combinations as antidepressants and related pharmacological properties can be determined by methods (1)-(3) below, which are described in Koe, B. et al. Journal of Pharmacology and Experimental Therapeutics, 226 (3), 686-700 (1983). Specifically, activity can be determined by studying (1) their ability to affect the efforts of mice to escape from a swim-tank by the Porsolt mouse “behavior despair” test, (2) their ability to potentiate 5-hydroxytryptophan-induced behavioral symptoms in mice in vivo, and (3) their ability to block the uptake of serotonin, norepinephrine, dopamine or a combination thereof by synaptosomal rat brain cells in vitro. The ability of the active combinations to counteract reserpine hypothermia in mice in vivo can be determined according to the methods described in U.S. Pat. No. 4,029,731. The activity of the active combinations as antidepressants and related pharmacological properties also can be determined by methods (4)-(8) below. Specifically, activity can be determined by studying (4) their ability to reverse the stress-induced decrease in sucrose intake in rodents described in Papp, M. et al., European Journal of Pharmacology, 261, 141-147 (1994), (5) learned helplessness paradigm described in Martin P et al., Life Sciences, 48, 2505-2511 (1991), (6) reversing the behavioral deficits of olfactory bulbectomized rats described in Broekkamp C L et al., Pharmacology, Biochemistry and Behavior, 13, 643-646 (1980), (7) increasing down-regulation or desensitization of beta-adrenergic receptors described in Mishra R. et al., Neuropharmacology, 19, 983-987 (1980), and (8) increasing extracellular levels of serotonin, norepinephrine, and/or dopamine in the prefrontal cortex of freely-moving rodents by in vivo dialysis described in Millan M J et al., European Journal of Neuroscience, 12, 1079-1095 (2000).

Activity of the active combinations in the treatment of anxiety may be determined from lactate-induced panic-like responses in panic-prone rats as described in Shekhar, A. and Keim S. R., J. Neurosci, 1997, 17:9726-9735, and Shekhar, A. and Keim, S. R., Neuropharmacol., 2000, 39:1139-1146. Activity of the active combinations in the treatment of anxiety may also be determined from anxiety screens such as those that include various derivations of conflict models or punishment models in rodents as described by J. L. Howard and G. T. Pollard in Psychopharmacologg of Anxiolytics and Antidepressants, (ED) SE File (1991), Pergamon press Inc. (New York), pp.131-153.

The pharmaceutical compositions described herein may be prescription pharmaceutical compositions or over-the-counter pharmaceutical compositions. As used herein, a “prescription pharmaceutical composition” is a composition which is effective to deliver an active compound to a human as prescribed by a physician. An “over-the-counter pharmaceutical composition” is a composition which is effective to deliver an active compound to a human which does not require a prescription from a physician in order to be administered to the human.

The pharmaceutical compositions described herein may be formulated in a conventional manner using one or more pharmaceutically acceptable carriers. Thus, the active combinations of this invention may be formulated for oral, buccal, intranasal, parenteral (for example, intravenous, intramuscular or subcutaneous), sublingual or rectal administration, or may be in the form of a patch, or in a form suitable for administration by inhalation or insufflation, and may be administered orally, buccally, intranasally, parenterally (for example, intravenously, intramuscularly or subcutaneously) or rectally or by inhalation or insulation.

For oral administration, the pharmaceutical compositions may take the form of, for example, tablets or capsules prepared by conventional means with pharmaceutically acceptable excipients such as binding agents, including pregelatinized maize starch, polyvinylpyrrolidone or hydroxypropyl methylcellulose; fillers, including lactose, microcrystalline cellulose or calcium phosphate; lubricants, including magnesium stearate, talc or silica; disintegrants, including potato starch or sodium starch glycolate; or wetting agents, including sodium lauryl sulphate. The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, including sorbitol syrup, methyl cellulose or hydrogenated edible fats; emulsifying agents, including lecithin or acacia, non-aqueous vehicles, including almond oil, oily esters or ethyl alcohol; and preservatives, including methyl or propyl p-hydroxybenzoates or sorbic acid.

For buccal administration, the composition may take the form of tablets or lozenges formulated in conventional manner.

The active compounds used in formulating the pharmaceutical composition of this invention may be formulated for parenteral administration by injection, including using conventional catheterization techniques or infusion. Formulations for injection may be presented in unit dosage form, for example, in ampoules or in multi-dose containers, with an added preservative.

The compositions containing the active compounds may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, for example, sterile pyrogen-free water, before use.

The active compounds used in formulating the pharmaceutical composition of this invention may also be formulated in rectal compositions such as suppositories or retention enemas, for example, containing conventional suppository bases such as cocoa butter or other glycerides.

For intranasal administration or administration by inhalation, the active compounds used in formulating the pharmaceutical compositions of this invention are conveniently delivered in the form of a solution or suspension from a pump spray container that is squeezed or pumped by the patient or as an aerosol spray presentation from a pressurized container or a nebulizer, with the use of a suitable propellant, for example, dichlorodifluoromethane, trichlorofluoromethane, dichlorotetrafluoroethane, carbon dioxide or other suitable gas. In the case of a pressurized aerosol, the unit dose may be determined by providing a valve to deliver a metered amount. The pressurized container or nebulizer may contain a solution or suspension of the active compounds. Capsules and cartridges, made, for example, from gelatin, for use in an inhaler or insufflator may be formulated containing a powder mix of a compound of this invention and a suitable powder base such as lactose or starch.

The norepinephrine reuptake inhibitors and the serotonin reuptake inhibitors used in formulating the pharmaceutical compositions of this invention may be administered alone or preferably together with pharmaceutically acceptable carriers by any of the routes previously indicated, and such administration may be carried out in both single and multiple doses. More particularly, the active combination can be administered in a wide variety of different dosage forms, i.e., the active combination may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, oral pharmaceutical formulations containing the active combination may be suitably sweetened and/or flavored by means of various agents of the type commonly employed for such purposes.

The amounts of a) the serotonin reuptake inhibitor or pharmaceutically acceptable salt thereof, and b) the norepinephrine reuptake inhibitor or pharmaceutically acceptable salt thereof are preferably amounts such that the combination of a) and b) is effective in treating the disorder or condition. The amount of the serotonin reuptake inhibitor or pharmaceutically acceptable salt thereof is preferably an amount effective in enhancing serotonergic neurotransmission in a mammal. The amount of the norepinephrine reuptake inhibitor or pharmaceutically acceptable salt thereof is preferably an amount effective in enhancing noradrenergic neurotransmission in a mammal. In one preferred embodiment, the amount of the serotonin reuptake inhibitor is an amount sufficient for dopamine reuptake inhibition. Affinity for the sertraline transporter (SERT) and for DAT transporter is determined by the amount of the β-CIT ligand that is displaced. The extent of dopamine reuptake inhibition, measured by transporter displacement using β-CIT in combination with extracellular dopamine increase, is greater than the extent of placebo.

The pharmaceutical compositions of the inventions achieve occupancy of the serotonin transporter and of the norepinephrine transporter by combining a serotonin reuptake inhibitor or pharmaceutically acceptable salt thereof with a norepinephrine reuptake inhibitor or pharmaceutically acceptable salt thereof. In one exemplary embodiment of the invention, the serotonin reuptake inhibitor or pharmaceutically acceptable salt thereof may be present in an amount sufficient to displace at least about 45% β-CIT from the serotonin transporter as assessed by SPECT imaging. The norepinephrine reuptake inhibitor or pharmaceutically acceptable salt thereof may be present in an amount such that the norepinephrine transporter has at least 50% occupancy, as an example at least 75% occupancy, as another example between 75% and 90% occupancy, as another example at least 90% occupancy, as another example between 90% and 100% occupancy, including 100% occupancy. In an exemplary embodiment, at least 75% occupancy of the norepinephrine transporter is maintained until the administration of a successive unit dose or unit dosage form of the composition of the invention. As used herein, “occupancy of the norepinephrine transporter” is intended to refer to occupancy of all norepinephrine transporters. Similarly, for example, “at least 75% occupancy” of the norepinephrine transporter is intended to mean that all norepinephrine transporters have an occupancy of at least 75%. Similarly, as another example, “at least 90% occupancy” of the norepinephrine transporter is intended to mean that all norepinephrine transporters have an occupancy of at least 90%. Similarly, as used herein, “an amount sufficient to displace at least about 45% β-CIT from the serotonin transporter” is an amount sufficient to displace at least about 45% β-CIT from all serotonin transporters.

In another embodiment of the invention, the serotonin reuptake inihibitor is present in an amount sufficient for dopamine reuptake inhibition. An amount sufficient for dopamine reuptake inhibition is an amount sufficient to displace at least about 15% β-CIT from the dopamine transporter, wherein “an amount sufficient to displace at least about 15% β-CIT from the dopamine transporter” is intended to mean an amount sufficient to displace at least about 15% β-CIT from all dopamine transporters. For example, the amount sufficient for dopamine reuptake inhibition may be an amount sufficient to displace about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, and 40% β-CIT from the dopamine transporter. When sertraline is used as the serotonin reuptake inihibitor, the amount sufficient to displace at least about 15% β-CIT from the dopamine transporter is about 150 mg.

Binding data are shown in Tables 1-3 herein. During periods 1, 2, and 3, there were statistically significant differences among the treatment groups for each brain site shown in the tables (p≦0.040). For midbrain and diencephalon, the mean [¹²³I] β-CIT binding for sertraline 25 mg and sertraline 50 mg were significantly less than for placebo. For striatum, the mean [¹²³I] β-CIT binding for sertraline 25 mg and sertraline 50 mg were significantly greater than for placebo. There were no statistically significant period or carryover effects (p≧0.052). The comparisons of the treatments during successive randomization periods 1, 2, and 3 are summarized in Table 1 below.

TABLE 1
Summary of Analysis of [¹²³ I] β-CIT Binding for Periods 1, 2, and 3

| | | | | | | 90% Confidence | |
|---|---|---|---|---|---|---|---|
| | Treatment | | Means (LS means) | | | Limits | |
| Site | Test | Reference | Test | Reference | Difference | Lower | Upper |
| Midbrain | Sertraline | Placebo | 0.91 | 1.49 | −0.58 | −1.03 | −0.13 |
| | 25 mg | | | | | | |
| | Sertraline | Placebo | 0.60 | 1.49 | −0.89 | −1.34 | −0.45 |
| | 50 mg | | | | | | |
| Diencephalon | Sertraline | Placebo | 1.91 | 3.42 | −1.51 | −2.23 | −0.80 |
| | 25 mg | | | | | | |
| | Sertraline | Placebo | 1.58 | 3.42 | −1.84 | −2.56 | −1.13 |
| | 50 mg | | | | | | |
| Striatum | Sertraline | Placebo | 10.06 | 9.44 | 0.62 | 0.26 | 0.98 |
| | 25 mg | | | | | | |
| | Sertraline | Placebo | 9.83 | 9.44 | 0.39 | 0.03 | 0.75 |
| | 50 mg | | | | | | |

For the comparison of periods 1, 2, 3, and 4, there were statistically significant differences among the treatment groups for each site (p≦0.007). For midbrain and diencephalon, the mean [¹²³I] β-CIT binding for sertraline 25 mg, sertraline 50 mg, and sertraline 150 mg were significantly less than for placebo. For striatum, the mean [¹²³I]β-CIT binding for sertraline 150 mg was significantly less than for placebo. The Least Square Means for sertraline 25 mg and sertraline 50 mg were greater than the mean for placebo but these differences were not statistically significant. The comparisons of the treatments during successive randomization periods 1, 2, 3, and 4 are summarized in Table 2 below.

TABLE 2
Summary of Analysis of [¹²³ I] β-CIT Binding for Periods 1, 2, 3, and 4

| | | | | | | 90% Confidence | |
|---|---|---|---|---|---|---|---|
| | Treatment | | Means (LS means) | | | Limits | |
| Site | Test | Reference | Test | Reference | Difference | Lower | Upper |
| Midbrain | Sertraline | Placebo | 0.93 | 1.45 | −0.52 | −0.84 | −0.20 |
| | 25 mg | | | | | | |
| | Sertraline | Placebo | 0.63 | 1.45 | −0.82 | −1.14 | −0.50 |
| | 50 mg | | | | | | |
| | Sertraline | Placebo | 0.77 | 1.45 | −0.68 | −0.99 | −0.36 |
| | 150 mg | | | | | | |
| Diencephalon | Sertraline | Placebo | 1.88 | 3.23 | −1.35 | −1.97 | −0.72 |
| | 25 mg | | | | | | |
| | Sertraline | Placebo | 1.80 | 3.23 | −1.43 | −2.05 | −0.80 |
| | 50 mg | | | | | | |
| | Sertraline | Placebo | 1.62 | 3.23 | −1.60 | −2.23 | −0.98 |
| | 150 mg | | | | | | |
| Striatum | Sertraline | Placebo | 10.04 | 9.32 | 0.72 | −0.16 | 1.60 |
| | 25 mg | | | | | | |
| | Sertraline | Placebo | 9.97 | 9.32 | 0.64 | −0.23 | 1.52 |
| | 50 mg | | | | | | |
| | Sertraline | Placebo | 8.17 | 9.32 | −1.15 | −2.03 | −0.28 |
| | 150 mg | | | | | | |

Table 3 shows the mean binding and mean percent displacement for [¹²³I]β-CIT in the midbrain, diencephalon, and striatum based on images collected with the Prism-XP camera. These data show that sertraline at 150 mg binds to the dopamine transporter.

TABLE 3
Mean Binding and Mean Percent Displacement of [¹²³I] β-CIT for Midbrain,
Diencephalon, and Striatum Based on images collected with the Prism-XP camera

| | No. of subjects = 6 | | | | |
|---|---|---|---|---|---|
| | | | Sertraline | | |
| | Baseline | Placebo | 25 mg | 50 mg | 150 mg |
| Midbrain | | | | | |
| Mean ± SD | 1.65 ± 0.39 | 1.45 ± 0.38 | 0.93 ± 0.64 | 0.63 ± 0.36 | 0.77 ± 0.31 |
| Mean Percent | — | — | −46.06 ± 26.72 | −61.17 ± 24.78 | −53.69 ± 10.89 |
| Dispacement ± SD | | | | | |
| Diencephalon | | | | | |
| Mean ± SD | 3.28 ± 0.96 | 3.23 ± 1.2 | 1.88 ± 1.4 | 1.8 ± 0.45 | 1.62 ± 0.71 |
| Mean Percent | — | — | −46.29 ± 23.97 | −44.12 ± 9.23 | −49.41 ± 16.02 |
| Dispacement ± SD | | | | | |
| Striatum | | | | | |
| Mean ± SD | 9.79 ± 3.48 | 9.32 ± 3.48 | 10.04 ± 3.85 | 9.97 ± 3.3 | 8.17 ± 2.66 |
| Mean Percent | — | — | 2.4 ± 6.81 | 2.66 ± 7.4 | −15.27 ± 14.5 |
| Dispacement ± SD | | | | | |

Baseline = Value on Day 0. The displacement percentages are based on the baseline values

An exemplary daily dose of a serotonin reuptake inhibitor in a pharmaceutical composition of this invention for oral, parenteral, rectal or buccal administration to the average adult human for the treatment of the conditions referred to above is from about 1 mg to 300 mg of serotonin reuptake inhibitor per unit dose administered 1 to 3 times per day, such as 25 mg to about 300 mg of sertraline, preferably from about 50 mg to about 200 mg of sertraline per unit dose which could be administered, for example, 1 to 3 times per day, or such as about 5 mg to about 80 mg of fluoxetine per unit dose, preferably from about 10 mg to about 40 mg of fluoxetine per unit dose which could be administered, for example, 1 to 3 times per day, or such as about 50 mg to about 300 mg of fluvoxamine per unit dose, preferably 100 mg to 200 mg of fluvoxamine per unit dose, which could be administered, for example, 1 to 3 times per day, or such as about 5 mg to about 30 mg of escitalopram, preferably about 10 mg to about 20 mg of escitalopram, which could be administered, for example, 1 to 3 times per day, or such as about 10 mg to about 60 mg of citalopram, preferably about 20 mg to about 40 mg of citalopram, which could be administered, for example, 1 to 3 times per day, or such as about 10 to about 50 mg of paroxetine, preferably about 10 mg to about 40 mg of paroxetine, which could be administered, for example, 1 to 3 times per day.

An exemplary daily dose of a serotonin reuptake inihibitor in a pharmaceutical composition of this invention for oral, parenteral, rectal or buccal administration to the average adult human for the treatment of the conditions referred to above, wherein the dose is sufficient for dopamine reuptake inhibition, is 150 mg or more of the serotonin reuptake inhibitor per unit dose per day. If sertraline is used as the serotonin reuptake inhibitor, amounts of about 150, 160, 170, 180, 190, 200, 210, 220, 230, 240, 250, 260, 270, 280, 290, 300, 310, 320, 330, 340, and 350 mg per unit dose per day are exemplary amounts that are sufficient for dopamine reuptake inhibition. As additional examples, the amount of sertraline sufficient for dopamine reuptake inhibition may range from about 150 mg to about 350 mg; about 150 mg to about 200 mg; from about 170 mg to about 340 mg; from about 190 mg to about 330 mg; from about 200 mg to about 310 mg; from about 210 mg to about 300 mg; from about 220 mg to about 290 mg; from about 230 mg to about 280 mg; from about 240 mg to about 270 mg; from about 240 mg to about 250 mg; from about 250 mg to about 260 mg; from about 260 mg to about 270 mg; from about 270 mg to about 280 mg; from about 280 mg to about 290 mg; and from about 290 mg to about 300 mg.

An exemplary daily dose of the norepinephrine reuptake inhibitor in a pharmaceutical composition of this invention for oral, parenteral, rectal or buccal administration to the average adult human for the treatment of the conditions referred to above is from about 1 to 300 mg of norepinephrine reuptake inhibitor per unit dose, such as about 1 mg to about 30 mg of racemic reboxetine, preferably from about 4 mg to about 16 mg of racemic reboxetine per unit dose which could be administered, for example 1 to 3 times per day. Another exemplary daily dose of the norepinephrine reuptake inhibitor is from about 1 mg to about 15 mg of [S,S]-reboxetine, preferably from about 2 mg to about 8 mg of [S,S]-reboxetine per unit dose which could be administered, for example 1 to 3 times per day. Another exemplary daily dose of the norepinephrine reuptake inhibitor is from about 150 mg to about 300 mg of amoxapine per unit dose, preferably between 200 mg and 275 mg of amoxapine per unit dose, which could be administered, for example 1 to 3 times per day. Another exemplary daily dose of the norepinephrine reuptake inhibitor is from about 25 mg to about 200 mg of maprotiline per unit dose, preferably between 50 mg and 150 mg of maprotiline per unit dose, which could be administered, for example 1 to 3 times per day.

An exemplary dose ratio by weight of a serotonin reuptake inhibitor to a norepinephrine reuptake inhibitor combination formulation for oral, parenteral or buccal administration to the average adult human for the treatment of the conditions referred to above is from about 0.00005 to about 20,000, preferably from about 0.25 to about 2,000.

In an exemplary embodiment of the invention, a serotonin reuptake inhibitor is present in a composition of the invention in an amount ranging from about 0.5% to about 90% by weight of the total composition, preferably from about 5% to about 80% by weight of the total composition; and a norepinenephrin reuptake inhibitor is present in the composition of the invention in an amount ranging from about 0.5% to about 90% by weight of the total composition, preferably from about 5% to about 80% by weight of the total composition. The ratio by weight of the amount of the serotonin reuptake inhibitor to the amount of the norepinephrine reuptake inhibitor preferably ranges from 20:1 to 1:20, more preferably from 10:1 to 1:10.

Aerosol combination formulations for treatment of the conditions referred to above in a mammal, such as an average adult human are preferably arranged so that each metered dose or “puff” of aerosol contains from about 100 μg to about 30,000 μg of the norepinephrine reuptake inhibitor, preferably from about 250 μg to about 1,000 μg of norepinephrine reuptake inhibitor, and from about 1,000 μg to about 30,000 μg of a serotonin reuptake inhibitor, preferably from about 5,000 μg to about 20,000 μg. Administration may be once or several times daily, for example 1, 3, 4 or 8 times, giving for example, 1, 2 or 3 doses each time.

It should be understood that the present invention is not limited to the embodiments described herein. Numerous modifications can be made by one skilled in the art having the benefits of the teachings given here. Such modifications should be taken as being encompassed within the scope of the present invention as set forth in the appended claims.

1-27. (cancelled).

28. A method for treating a disorder or condition that can be treated by enhancing serotonergic neurotransmission in a mammal, noradrenergic neurotransmission in a mammal, or a combination thereof, comprising administering to a mammal in need of such treatment: (i) at least one serotonin reuptake inhibitor or pharmaceutically acceptable salt thereof; and (ii) at least one norepinephrine reuptake inhibitor or pharmaceutically acceptable salt thereof, wherein the norepinephrine reuptake inhibitor is selected from the group consisting of Structure II, Structure III, and Structure IV, wherein:
(A) Structure II has the formula 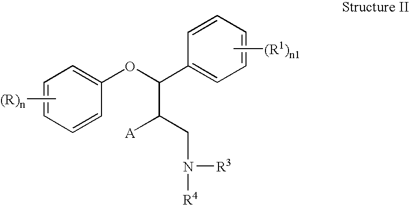
wherein n and n₁ are, independently, 1, 2 or 3; each or the groups R and R¹, which may be the same or different, is hydrogen; halogen; halo-C₁-C₆ alkyl: hydroxy: C₁-C₆ alkoxy: C₁-C₆ alkyl optionally substituted with C₁-C₆ alkyl or halogen: aryl-C₁-C₆ alkyl optionally substituted with C₁-C₆ alkyl or halogen; aryl-C₁-C₆ alkoxy optionally substituted with C₁-C₆ alkyl or halogen: —NO₂; —NR⁵R⁶ wherein R⁵ and R⁶ are, independently, hydrogen or C₁-C₆ alkyl, or two adjacent R groups or two adjacent R¹ groups, taken together, form the —O—CH₂—O— group;
A is H or OR²;
R² is hydrogen; C₁-C₁₂ alkyl optionally substituted with C₁-C₆ alkyl or halogen, or aryl-C₁-C₆ alkyl;
each of the groups R³ and R⁴, which may be identical or different, is hydrogen, C₁-C₆ alkyl optionally substituted with C₁-C₆ alkyl or halogen, C₂-C₄ alkenyl, C₂-C₄ alkynyl, aryl-C₁-C₄ alkyl optionally substituted with C₁-C₆ alkyl or halogen, C₃-C₇ cycloalkyl optionally substituted with C₁-C₆ alkyl or halogen, or R³ and R⁴ with the nitrogen atom to which they are bound form a pentatomic or hexatomic saturated or unsaturated, optionally substituted with C₁-C₆ alkyl or halogen, heteromonocyclic group optionally containing other heteroatoms selected from the group consisting of O,S and N;
or R² and R⁴, taken together, form the —CH₂—CH₂-group;
(B3) Structure III has the formula 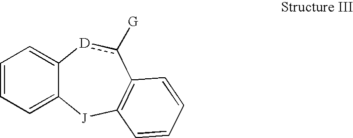
wherein D is N or CR⁹, where R⁹ is hydrogen. C₁-C₆ alkyl optionally substituted with C₁-C₆ alkyl or halogen, C₂-C₄ alkenyl, C₂-C₄ alkynyl, aryl-C₁-C₄ alkyl optionally substituted with C₁-C₆ alkyl or halogen, or C₃-C₇ cycloalkyl optionally substituted with C₁-C₆ alkyl or halogen; G is NR⁷R⁸, wherein each of R⁷ and R⁸ is independently hydrogen, C₁-C₆ alkyl optionally substituted with C₁-C₆ alkyl or halogen, C₂-C₄ alkenyl, C₂-C₄ alkynyl, aryl-C₁-C₄ alkyl optionally substituted with C₁-C₆ alkyl or halogen, or C₃-C₇ cycloalkyl optionally substituted with C₁-C₆ alkyl or halogen; or R⁷ and R⁸ taken together with the nitrogen atom to which they are bound form a pentatomic or hexatomic, saturated or unsaturated, optionally substituted with C₁-C₆ alkyl or halogen heteromonocyclic group optionally containing one or more further additional heteroatoms selected from the group consisting of O,S and N; the bond between D and the ring carbon bonded to G is single or double; and J is O or L, where L is 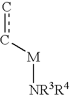
where the bond between the ring carbon of L and the carbon of L bonded to M is single or double; M is a Cₙ alkylene chain, where n is between 1 and 3; and each of R¹³ and R¹⁴ is independently hydrogen, C₁-C₆ alkyl optionally substituted with C₁-C₆ alkyl or halogen, C₂-C₄ alkenyl, C₁-C₄ alkynyl, aryl-C₁-C₄ alkyl optionally substituted with C₁-C₆ alkyl or halogen, C₃-C₇ cycloalkyl optionally substituted with C₁-C₆ alkyl or halogen, or R¹³ and R¹⁴ taken together with the nitrogen atom to which they are bound form a pentatomic or hexatomic, saturated or unsaturated, optionally substituted with C₁-C₆ alkyl or halogen heteromonocyclic group optionally containing one or more further additional heteroatoms selected from the group consisting of O,S and N; and
(C) Structure IV has the formula 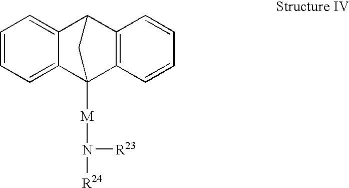
wherein M is a Cₙ alkylene chain, where n is between 1 and 3; and each of R²³ and R²⁴ is independently hydrogen, C₁-C₆ alkyl optionally substituted with C₁-C₆ alkyl or halogen, C₂-C₄ alkenyl, C₂-C₄ alkynyl, aryl-C₁-C₄ alkyl optionally substituted with C₁-C₆ alkyl or halogen, C₃-C₇ cycloalkyl optionally substituted with C₁-C₆ alkyl or halogen, or R²³ and R²⁴ taken together with the nitrogen atom to which they are bound form a pentatomic or hexatomic, saturated or unsaturated, optionally substituted with C₁-C₆ alkyl or halogen heteromonocyclic group optionally containing one or more further additional heteroatoms selected from the group consisting of O,S and N,
wherein the norepinephrine reuptake inhibitor or pharmaceutically acceptable salt thereof is present in an amount such that all norepinephrine transporters have an occupancy of at least 50% and the serotonin reuptake inhibitor or pharmaceutically acceptable salt thereof is present in an amount sufficient to displace at least about 45% β-CIT from the serotonin transporter as assessed by SPECT imaging.

29. A method for treating depression in a mammal, comprising administering to a mammal in need of such treatment: (i) at least one serotonin reuptake inhibitor or pharmaceutically acceptable salt thereof; and (ii) at least one norepinephrine reuptake inhibitor or pharmaceutically acceptable salt thereof, wherein the norepinephrine reuptake inhibitor is selected from the group consisting of Structure II, Structure III, and Structure IV as defined in claim 28.

30. The method of claim 29, wherein the disorder or condition is depression selected from the group consisting of depression in Parkinson's patients, Postmyocardial Infarction depression, depression in patients with human immunodeficiency virus (HIV), Subsyndromal Symptomatic depression, depression in infertile women, pediatric depression, major depression, single episode depression, recurrent depression, child abuse induced depression, post partum depression, DSM-IV major depression, treatment-refractory major depression, severe depression, psychotic depression, post-stroke depression, neuropathic pain, manic depressive illness, manic depressive illness with mixed episodes, manic depressive illness with depressive episodes, bipolar depression BP I, and bipolar depression BP II, melancholy, and major depression with dysthymia.

31. The method of claim 29, wherein the serotonin reuptake inhibitor is sertraline which is present in an amount sufficient for dopamine reuptake inhibition.

32. (cancelled)

33. The method of claim 29, wherein the norepinephrine reuptake inhibitor or pharmaceutically acceptable salt thereof is present in an amount such that all norepinephrine transporters have an occupancy of at least 75%.

34. The method of claim 29, wherein the serotonin reuptake inhibitor and the norepinephrine reuptake inhibitor are administered together in a pharmaceutical composition.

35. The method of claim 29, wherein the norepinephrine reuptake inhibitor is [S,S]-reboxetine administered in one or more unit doses each comprising about 1 to about 15 mg of [S,S]-reboxetine.

36. The method of claim 29, wherein the norepinephrine reuptake inhibitor is racemic reboxetine administered in one or more unit doses each comprising about 1 to about 30 mg of racemic reboxetine.

37. The method of claim 29, wherein the serotonin reuptake inhibitor is sertraline or a pharmaceutically acceptable salt thereof; the norepinephrine reuptake inhibitor is [S,S]-reboxetine or a pharmaceutically acceptable salt thereof, wherein sertraline is present in an amount sufficient for dopamine reuptake inhibition, wherein the amount of sertraline is from about 150 mg to about 200 mg.

38. The method of claim 31, wherein sertraline is used in an amount ranging from about 150 mg to about 350 mg.

39. The method of claim 31, wherein sertraline is used in an amount ranging from about 170 mg to about 340 mg.

40. The method of claim 31, wherein sertraline is used in an amount ranging from about 190 mg to about 330 mg.

41. The method of claim 31, wherein sertraline is used in an amount ranging from about 200 mg to about 310 mg.

42. The method of claim 31, wherein sertraline is used in an amount ranging from about 210 mg to about 300 mg.

43. The method of claim 31, wherein sertraline is used in an amount ranging from about 220 mg to about 290 mg.

44. The method of claim 31, wherein sertraline is used in an amount ranging from about 230 mg to about 280 mg.

45. The method of claim 31, wherein sertraline is used in an amount ranging from about 240 mg to about 270 mg.

46. The method of claim 31, wherein sertraline is used in an amount ranging from about 240 mg to about 250 mg.

47. The method of claim 31, wherein sertraline is used in an amount ranging from about 250 mg to about 260 mg.

48. The method of claim 31, wherein sertraline is used in an amount ranging from about 260 mg to about 270 mg.

49. The method of claim 31, wherein sertraline is used in an amount ranging from about 270 mg to about 280 mg.

50. The method of claim 31, wherein sertraline is used in an amount ranging from about 280 mg to about 290 mg.

51. The method of claim 31, wherein sertraline is used in an amount ranging from about 290 mg to about 300 mg.

52-56. (cancelled)

57. Use of (i) at least one serotonin reuptake inhibitor or pharmaceutically acceptable salt thereof and (ii) at least one norepinephrine reuptake inhibitor or pharmaceutically acceptable salt thereof for preparing a medicament for treating depression in a mammal, wherein the norepinephrine reuptake inhibitor is selected from the group consisting of Structure II, Structure III, and Structure IV as defined in claim 28.

58-64. (cancelled)

65. A composition comprising (i) at least one serotonin reuptake inhibitor or pharmaceutically acceptable salt thereof, wherein the at least one serotonin reuptake inhibitor is selected from the group consisting of sertraline, fluoxetine and fluvoxamine; and (ii) at least one norepinephrine reuptake inhibitor or pharmaceutically acceptable salt thereof, wherein the at least one norepinephrine reuptake inhibitor is selected from the group consisting of racemic reboxetine, [S,S]-reboxetine, amoxapine, and maprotiline,
wherein the norepinephrine reuptake inhibitor or pharmaceutically acceptable salt thereof is present in an amount such that all norepinephrine transporters have an occupancy of at least 50% and the serotonin reuptake inhibitor or pharmaceutically acceptable salt thereof is present in an amount sufficient to displace at least about 45% β-CIT from the serotonin transporter as assessed by SPECT imaging.

66. The composition of claim 65, wherein the at least one serotonin reuptake inhibitor is sertraline and the at least one norepinephrine reuptake inhibitor is [S,S]-reboxetine.

67. The composition of claim 65, wherein the norepinephrine reuptake inhibitor is [S,S]-reboxetine administered in one or more unit doses each comprising about 1 to about 15 mg of [S,S]-reboxetine.

68. The composition of claim 65, wherein the norepinephrine reuptake inhibitor is racemic reboxetine administered in one or more unit doses each comprising about 1 to about 30 mg of racemic reboxetine.

69. The composition of claim 65, wherein the serotonin reuptake inhibitor is sertraline which is present in an amount sufficient for dopamine reuptake inhibition.

70. A composition consisting essentially of (i) at least one serotonin reuptake inhibitor or pharmaceutically acceptable salt thereof, wherein the at least one serotonin reuptake inhibitor is selected from the group consisting of sertraline, fluoxetine and fluvoxamine; and (ii) at least one norepinephrine reuptake inhibitor or pharmaceutically acceptable salt thereof, wherein the at least one norepinephrine reuptake inhibitor is selected from the group consisting of racemic reboxetine, [S,S]-reboxetine, amoxapine, and maprotiline,
wherein the norepinephrine reuptake inhibitor or pharmaceutically acceptable salt thereof is present in an amount such that all norepinephrine transporters have an occupancy of at least 50% and the serotonin reuptake inhibitor or pharmaceutically acceptable salt thereof is present in an amount sufficient to displace at least about 45% O-CIT from the serotonin transporter as assessed by SPECT imaging.

71. The composition of claim 70, wherein the at least one serotonin reuptake inhibitor is sertraline and the at least one norepinephrine reuptake inhibitor is [S,S]-reboxetine.

72. The composition of claim 70, wherein the serotonin reuptake inhibitor is sertraline which is present in an amount sufficient for dopamine reuptake inhibition.

73. A composition for treating a disorder or condition that can be treated by enhancing serotonergic neurotransmission in a mammal, dopaminergic transmission in a mammal, noradrenergic neurotransmission in a mammal, or a combination thereof, the composition comprising (i) sertraline or a pharmaceutically acceptable salt thereof, and optionally (ii) a norepinephrine reuptake inhibitor or a pharmaceutically acceptable salt thereof, wherein the norepinephrine reuptake inhibitor is selected from the group consisting of Structure II, Structure III, and Structure IV as defined in claim 28, wherein sertraline is present in an amount sufficient for dopamine reuptake inhibition.

74. A method for treating a disorder or condition that can be treated by enhancing serotonergic neurotransmission in a mammal, dopaminergic transmission in a mammal, noradrenergic neurotransmission in a mammal, or a combination thereof, the method comprising administering to a mammal in need of such treatment (i) sertraline or a pharmaceutically acceptable salt thereof, and optionally (ii) a norepinephrine reuptake inhibitor or a pharmaceutically acceptable salt thereof, wherein the norepinephrine reuptake inhibitor is selected from the group consisting of Structure II, Structure III, and Structure IV as defined in claim 28, wherein sertraline is present in an amount sufficient for dopamine reuptake inhibition.

75. A composition comprising sertraline or a pharmaceutically acceptable salt thereof and optionally a norepinephrine reuptake inhibitor selected from the group consisting of racemic reboxetine, [S,S]-reboxetine, amoxapine, and maprotiline, or pharmaceutically acceptable salts thereof, wherein sertraline or a pharmaceutically acceptable salt thereof is present in an amount sufficient for dopamine reuptake inhibition.

76. The composition of claim 75, wherein the norepinephrine reuptake inhibitor is [S,S]-reboxetine or a pharmaceutically acceptable salt thereof.

77. The composition of claim 76, wherein sertraline is used in an amount ranging from about 150 mg to about 350 mg.

78. The composition of claim 76, wherein sertraline is used in an amount ranging from about 170 mg to about 340 mg.

79. The composition of claim 76, wherein sertraline is used in an amount ranging from about 190 mg to about 330 mg.

80. The composition of claim 76, wherein sertraline is used in an amount ranging from about 200 mg to about 310 mg.

81. The composition of claim 76, wherein sertraline is used in an amount ranging from about 210 mg to about 300 mg.

82. The composition of claim 76, wherein sertraline is used in an amount ranging from about 220 mg to about 290 mg.

83. The composition of claim 76, wherein sertraline is used in an amount ranging from about 230 mg to about 280 mg.

84. The composition of claim 76, wherein sertraline is used in an amount ranging from about 240 mg to about 270 mg.

85. The composition of claim 76, wherein sertraline is used in an amount ranging from about 240 mg to about 250 mg.

86. The composition of claim 76, wherein sertraline is used in an amount ranging from about 250 mg to about 260 mg.

87. The composition of claim 76, wherein sertraline is used in an amount ranging from about 260 mg to about 270 mg.

88. The composition of claim 76, wherein sertraline is used in an amount ranging from about 270 mg to about 280 mg.

89. The composition of claim 76, wherein sertraline is used in an amount ranging from about 280 mg to about 290 mg.

90. The composition of claim 76, wherein sertraline is used in an amount ranging from about 290 mg to about 300 mg.

91. The composition of claim 71, further comprising a 5-HT1B antagonist.

92. The composition of claim 91, wherein the 5-HT1B antagonist is elzasonan.

93. The composition of claim 92, wherein elzasonan is present in an amount between about 0.1 mg and about 10 mg.

94. The method of claim 74, further comprising administering to a mammal in need of such treatment elzasonan.

95. The composition of claim 66, wherein [S,S]-reboxetine is in the form of the fumarate, succinate, citrate or tartrate salt.

96. The composition of claim 67, wherein [S,S]-reboxetine is in the form of the fumarate, succinate, citrate or tartrate salt.

97. The composition of claim 68, wherein [S,S]-reboxetine is in the form of the fumarate, succinate, citrate or tartrate salt.

98. The composition of claim 69, wherein [S,S]-reboxetine is in the form of the fumarate, succinate, citrate or tartrate salt.

99. A composition for treating depression in a mammal, the composition comprising: (i) at least one serotonin reuptake inhibitor or pharmaceutically acceptable salt thereof; and (ii) at least one norepinephrine reuptake inhibitor or pharmaceutically acceptable salt thereof, wherein the at least one norepinephrine reuptake inhibitor is selected from the group consisting of Structure II, Structure III, and Structure IV as defined in claim 28.

100. The composition of claim 79, wherein the serotonin reuptake inhibitor is sertraline which is present in an amount sufficient for dopamine reuptake inhibition.

101. The composition of claim 79, wherein the serotonin reuptake inhibitor is sertraline or a pharmaceutically acceptable salt thereof, and the norepinephrine reuptake inhibitor is [S,S]-reboxetine or a pharmaceutically acceptable salt thereof, wherein sertraline is present in an amount sufficient for dopamine reuptake inhibition, wherein the amount of sertraline is from about 150 mg to about 200 mg.

102. The composition of claim 101, wherein [S,S]-reboxetine is in the form of the fumarate, succinate, citrate or tartrate salt.
